(19) United States
(12) Patent Application Publication
(10) Pub. No.: US 2008/0223379 A1
(43) Pub. Date: Sep. 18, 2008

(54) METHODS AND COMPOSITIONS FOR THE TREATMENT OR PROPHYLAXIS OF TENDON AND LIGAMENT INJURY

(75) Inventors: Gerhard Stuker, Bach (CH); Christian Fricker, Schofflisdorf (CH)

Correspondence Address: CHRISTENSEN, O'CONNOR, JOHNSON, KINDNESS, PLLC, 1420 FIFTH AVENUE, SUITE 2800, SEATTLE, WA, 98101-2347, US

(73) Assignee: DOBAVET GmbH, Bach (CH)

(21) Appl. No.: 12/045,600

(22) Filed: Mar. 10, 2008

(30) Foreign Application Priority Data
Mar. 12, 2007 (EP) 07 00 5084.4

(51) Int. Cl. A61B 19/00

(52) U.S. Cl. 128/898

(57) ABSTRACT

The present application relates to methods for the treatment and/or prophylaxis of tendon diseases by administration of calcium dobesilate (2,5-dihydroxybenzosulfonate).

### BACKGROUND

The invention relates to calcium dobesilate for the treatment and/or prophylaxis of tendon diseases and a pharmaceutical composition containing calcium dobesilate for the treatment and/or prophylaxis of tendon diseases.

The invention further relates to the use of calcium dobesilate for the preparation of a pharmaceutical composition for the treatment and/or prophylaxis of tendon diseases.

The invention further relates to a method for the treatment and prophylaxis of such tendon diseases, wherein an effective amount of the above-mentioned compound is administered to a mammal.

The invention further relates to the use of calcium dobesilate in combination with a platelet aggregation inhibitor for the preparation of a pharmaceutical composition for the treatment and/or prophylaxis of such tendon diseases. The tendon disease to be treated can be a tendinitis or can be caused by degenerative processes.

The previous use of calcium dobesilate for the treatment and prophylaxis of bone and joint diseases is described in EP-0 670 721 B1. U.S. Pat. No. 3,509,207 describes the use of calcium dobesilate as a hemostatic.

Strictly speaking, tendinitis is a tendon inflammation referring to the inflammatory changes of the tendon tissue. It often results in degenerative changes of the tissue affected, possibly including calcium deposits at a later time. In general, tendinitis can affect any tendon of the body. Since tendinitis is mainly caused by mechanical stress (sports), tendinitis affects in particular certain body regions, such as e.g. in the region of the shoulder, the tibia or the foot. Tendinitis can also occur in the context of inflammatory rheumatoid diseases (in particular Reiter's syndrome, spondylitis ankylosans, arthritis psoriatica).

The three most frequent forms of tendinitis in humans are:

1. tendinitis of the musculus tibialis anterior tendon

2. tendinitis calcarea (mostly shoulder joint affected)

3. tendinitis of the Achilles tendon

Present therapy methods of frequent forms of tendinitis 1. Therapy of tendinitis affecting the musculus tibialis anterior tendon consists in the immobilisation of lower leg and foot, in severe cases by means of a plaster bandage. The application of ice and antiphlogistic analgesics is also useful. In refractory cases, therapeutic local anaesthetics are applied in form of repeated infiltrations of a long-acting local anaesthetic around the tendon, in between also with the addition of cortisone.

2. The treatment of tendinitis calcarea includes, in the beginning, i.e. during the acute phase, the immobilisation of the shoulder (arm sling), subsequent physiotherapy, i.e. arm swings initially passive, then active. Cold packs are also useful. Medicinal treatment consists in the prescription of non-steroidal antirheumatics. If these measures do not suffice, therapeutic local anaesthetics are applied in form of repeated infiltration of a long-acting local anaesthetic, in between also with the addition of cortisone. Unfortunately, there are always cases in which a painful (chronic) tendinitis persists in spite of adequate treatment. A very useful method for treating this form of tendinitis is the continuous brachial plexus block wherein the local anaesthetic is injected without any pain via a catheter several times a day after the effect of the previous dose has worn off. Meanwhile, there has been scientific evidence that local anaesthetics also have an anti-inflammatory effect.

3. With tendinitis of the Achilles tendon, physical therapies, cooling ice packs, ultrasound therapy and constant current treatment and iontophoresis are applied. These are able to alleviate the problems to a certain degree, the time factor, however, persists. Often, a shoe insert in form of a heel wedge or an appropriate bandage is useful. If there are tendon tears, cortisone-free analgesics can be applied locally. A rupture of the Achilles tendon does not require surgical treatment, if the tendon ends are located sufficiently close to one another and the patient is advanced in age. Injection of cortisone preparations is advised against, since, in case a partial rupture of the Achilles tendon was not detected, the crystallisation of cortisone might cause a complete rupture of the tendon. If the tendon is highly calcified (a another possible cause for tendinitis of the Achilles tendon), scarred parts of the tendon may be surgically resected. Furthermore, tendinitis of the Achilles tendon can be treated with a specific pain therapy. Successful treatment necessarily includes both the treatment of pain and the treatment of the inflammation. Therapeutic local anaesthetics are particularly suitable for this purpose. Local infiltration with a local anaesthetic, however, is rather painful and, thus, is hardly appropriate for repeated standard application. In cases of chronic tendinitis of the Achilles tendon, repeated blockage of the nervus ischiadicus is advantageous, in refractory cases, continuous blockage via catheter is ideal.

### DETAILED DESCRIPTION

The present invention particularly relates to the use in veterinary medicine, in particular for horses.

In a preferred embodiment, the tendinitis is a tendinitis of the superficial digital flexor tendon of the horse. Due to its frequency, the tendinitis of the superficial digital flexor tendon is the tendon disease which has been investigated best. The superficial digital flexor tendon of the horse is an elastic structure the physiological functionality of which is limited under maximum stress. The biomechanical and biochemical reactions of the superficial digital flexor tendon to work and to injury and the healing processes are not completely known. However, recent results of scientific studies provide valuable information. Apparently, the tissue of the superficial digital flexor tendon matures early in ontogenesis. Once development is terminated, there are almost no possibilities for the tendon tissue to adjust to exceptional stress, for instance, by increased elasticity. Stress of the tendon always results in progressive tissue degeneration. Focal decrease of cells, degeneration of collagen fibrils, selective increase of the forces acting upon the fibrils and alterations of the non-collagenous tissue matrix become manifest mainly in the central middle part of the tendon at the level of the metacarpus.

The present standard strategies for treating tendinitis of the superficial digital flexor tendon of the horse, which aim at restoring a sport horse so that it can deliver maximum performance, show varying and on the whole unsatisfying results. Modern rehabilitation measures, if combined with regular ultrasound control examinations, are definitely equivalent to surgical measures and also more cost-effective. Recently, scientific concern focussed in particular on the pharmacological modulation of the healing processes at collagen structures. Various growth factors were studied as possible therapeutic means supporting the healing of tendon injuries.

According to the invention, it was found that treating tendon diseases such as tendinitis with calcium dobesilate results in the recovery of the affected tissue. This applies in particular to the issue of the various forms of Achilles tendon inflammations in human medicine and to the tendinitis of the superficial digital flexor tendon of the horse in veterinary medicine.

According to the invention, it was possible to establish that, due to the treatment, the affected tendons regained their original structure.

In addition to calcium dobesilate, the pharmacological compositions may contain platelet aggregation inhibitors such as acetylsalicylic acid (ASA) and/or benzopyrone compounds.

The invention relates the use of calcium dobesilate of the formula

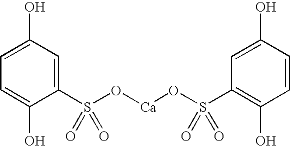

The preparation of this compound is described in U.S. Pat. No. 3,509,207.

In combination with calcium dobesilate, the following benzopyrones are suitable for the use according to the invention:

1,2-benzopyrone derivatives of the general formula I

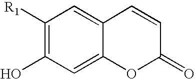

wherein R₁ is hydrogen, halogen or a hydroxy, sulfonyl, alkyl, hydroxyalkyl, acyloxy, alkoxy or benzyl group or a glycoside residue, and 1,4-benzopyrones of the general formula II

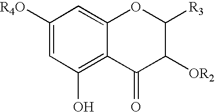

wherein R₂ and R₄ are, independently from each other, hydrogen, halogen or a hydroxy, sulfonyl, alkyl, hydroxyalkyl, acyloxy, alkoxy or benzyl group or a glycoside residue, and R₃ represents hydrogen or a phenyl residue of the general formula

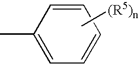

wherein R⁵ is a halogen atom, a hydroxy, sulfonyl, alkyl, hydroxyalkyl, alkyloxy, alkoxy group and n can have values between 0 to 3.

In combination with calcium dobesilate, the following benzopyrones are particularly preferred for the use according to the invention:

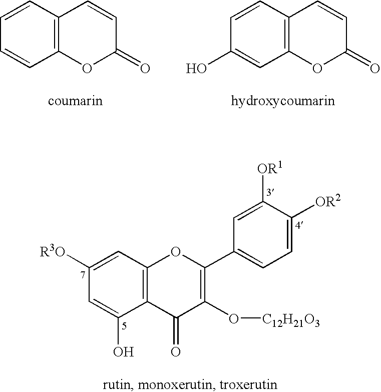

R¹=R²=R³=H; rutin

R¹=R²=H; R³=(CH₂)₂—OH monorutin

R¹=R²=R³=(CH₂)₂—OH troxerutin

as well as the compound diosnin

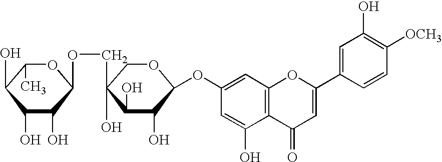

The above compounds can be produced in a manner known per se, see Beilstein III/IV, Vol. 18, p. 294 et seq. Furthermore, these compounds are commercially available as natural products.

In combination with calcium dobesilate, acetylsalicylic acid is particularly preferred for the use according to the invention:

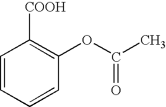

The above compound can be produced in a manner known to the person skilled in the art. Furthermore, these compounds are commercially available.

Moreover, the present invention provides pharmaceutical compositions comprising calcium dobesilate, optionally in combination with a further active agent according to the invention, optionally in admixture with adjuvants and excipients common in the field. The pharmaceutical compositions according to the invention can be formulated/produced according to standard methods and techniques known to the person skilled in the art; such as described e.g. in Remington's Pharmaceutical Sciences, 15^{th} edition, Mack Publishing Co., New Jersey (1991).

In this context, dosage forms for oral, parenteral (e.g. i.v., s.c., i.p., i.c., intra-thecal) and local (e.g. topic, rectal, vaginal, buccal, application in the eye or by inhalation) application are preferred.

Thus, the pharmaceutical compositions according to the invention can be present in particular as tablets (particularly also enteric coated tablets or tablets with modified release of the active agent), capsules (hard and soft gelatine capsules), pills, granulates, suppositories, ovula, ointments, creams, gels, plasters, TTS or also as emulsions, suspensions, solutions or reconstitutable powder (also for parenteral application).

The dosage depends on the patient's age, condition and weight as well as on the type of the application. As a rule, the daily dose of the active agent is 2 to 10 mg/kg body weight with oral administration, 1 to 10 mg/kg body weight with parenteral administration and 0.1 to 0.5 mg/kg body weight with topical administration.

Excipients used in the formulation can comprise filling agents (carriers), vehicles, diluents, solvents including monohydric alcohols such as ethanol, isopropanol and multihydric alcohols such as glycols, as well as edible oils such as soy oil, coconut oil, olive oil, safflower oil, cotton seed oil, oily esters such as ethyloleate, isopropylmyristate; binding agents, adjuvants, solubility mediators, thickening agents, stabilising agents, flow regulators, lubricants, buffers, emulgators, wetting agents, dispersants, sweeteners, colouring agents, aroma agents, coating agents, preservatives, antioxidants, disintegrants, softeners, sorption agents and/or retardation agents such as calcium phosphate, magnesium stearate, talcum, monosaccharides, disaccharides, starch, gelatine, cellulose, methyl cellulose, sodium carboxymethyl cellulose, dextrose, hydroxypropyl-β-cyclodextrin, polyvinylpyrrolidine, low-melting waxes and ion exchange resins.

The application forms thus obtained contain the active agent in an amount from 1 to 99% by weight, preferably in an amount of 20 to 99% by weight.

The following Examples confirm the effectiveness of the use according to the invention.

### EXAMPLE 1

Calcium dobesilate was administered with the food in a dose of 1400 mg per adult horse, twice a day each (corresponding to 4 mg/kg body weight/day). The therapy is carried out for four months. Therapy progress is documented sonographically after one month, after two months and after four months. During therapy, the horses are walked.

Sonographically, therapy progression always shows the following features: The tendon tissue damaged due to ruptured tendon fibrils and hematoma is degraded and substituted by directed collagen fibers, i.e. the tendon sections hypoechogenic at the beginning of therapy show a physiological structure and density of the tendon in the ultrasound image at the end of therapy.

While illustrative embodiments have been illustrated and described, it will be appreciated that various changes can be made therein without departing from the spirit and scope of the invention.

The embodiments of the invention in which an exclusive property or privilege is claimed are defined as follows:

1. A method for the treatment and/or prophylaxis of tendon diseases in a patient in need thereof comprising administering to the patient a composition comprising an effective amount of calcium dobesilate (2,5-dihydroxybenzosulfonate).

2. The method of claim 1, wherein the tendon disease is tendinitis.

3. The method of claim 1, wherein the patient is a human or nonhuman mammal.

4. The method of claim 3, wherein the patient is a horse.

5. The method of claim 4 wherein the tendon disease is tendinitis of the superficial digital flexor tendon of the horse.

6. The method of claim 1, wherein the composition further contains a platelet aggregation inhibitor.

7. The method of claim 6, wherein the platelet aggregation inibitor is selected from acetylsalicylic acid (ASA) and a benzopyrone compound.

8. The method of claim 7, wherein the platelet aggregation inibitor is a benzopyrone compound selected from coumarin, hydroxycoumarin, rutin, monoxerutin, troxerutin, and diosnin.

9. The method of claim 1, wherein the composition further comprises one or more pharmaceutically acceptable excipients.

10. The method of claim 1, wherein the composition is formulated for oral, rectal, topical, or parenteral administration.

11. The method of claim 10, wherein the composition is formulated for oral administration.
